(19) United States
(12) Patent Application Publication
(10) Pub. No.: US 2004/0242846 A1
(43) Pub. Date: Dec. 2, 2004

(54) Method for the preparation of molecularly uniform hyperpolymeric hemoglobins

(75) Inventor: Wolfgang Barnikol, Mainz (DE)

Correspondence Address: DAVID TOREN, ESQ., SIDLEY, AUSTIN, BROWN & WOOD, LLP, 787 SEVENTH AVENUE, NEW YORK, NY, 10019-6018, US

(73) Assignee: SanguBio Tech AG

(21) Appl. No.: 10/878,724

(22) Filed: Jun. 28, 2004

Related U.S. Application Data
(63) Continuation of application No. 08/869,406, filed on Jun. 5, 1997.
Application No. 08/869,406 is a continuation of application No. 08/455,426, filed on May 31, 1995, now abandoned.

(30) Foreign Application Priority Data
May 31, 1994 (DE) P 44 18 973.7

(51) Int. Cl. C07K 14/795

(52) U.S. Cl. 530/383

(57) ABSTRACT

A method of preparation of molecularly uniform hyperpolymeric hemoglobins includes subjecting a solution of hyperpolymeric hemoglobins with different molecular weights to an ultrafiltration process using a filter with a normal cut-off threshold of 10⁶ g/mol.

### RELATED APPLICATIONS

This application is a continuation of application Ser. No. 08/869,406 filed Jun. 5, 1997 which was a continuation of application Ser. No. 08/455,426 filed May 31, 1995 (now abandoned).

### BACKGROUND OF THE INVENTION

1. Field of Invention

The invention relates to a method for the preparation of molecularly uniform hyperpolymeric hemoglobins.

2. Description of the Prior Art

Hemoglobin is modified chemically, for example, by changing the oxygen affinity or by polymerizing the molecule, in order to make available blood substitute media, which can support the oxygen transport function in humans.

The blood substitute media can be infused, for example, in the case of a traffic accident with bleeding that is difficult to control and of accidents with loss of blood or in the event of a risk of infection (hepatitis, AIDS) as a substitute for suitable blood units, which are temporally not available, as well as when a person, in the cases named, is in a state of volume deficiency shock. It is possible that an oxygen-transferring blood substitute solution can breach a volume deficiency shock sooner than units of blood since, as is well known, erythrocytes are stiffened in the preserved blood and therefore have a decreased capillary permeability. An oxygen-transporting blood substitute is also more advantageous than preserved blood when there is the risk of a hyperintensive immunological reaction. It has been shown in animal experiments that a volume deficiency shock can be combated more effectively with oxygen-transferring blood substitute solutions than with simple plasma expanders (survey article: R. Pabst, Med. Klin. 72, (1977), 1555-1562). It is furthermore to be expected that chronic circulation disorders (for example, coronary, cerebral, peripheral) can be combated more effectively with the help of suitable polyhemoglobin solutions. Last but not least, oxygen deficiency states without a decrease in the circulation, such as chronic anemias, can also be combated with such solutions. It is estimated that the area of application for this indication is even ten times as large.

Various paths have already been taken to prepare oxygen-transferring blood substitute media, namely:

1. The use of emulsions of fluorinated hydrocarbons, in which the oxygen dissolves well (this subject is surveyed in: Hirlinger et al., Anaesthesist, 31, (1982), 660-666). This method, however, has the disadvantage that tissue reactions occur when fluorinated hydrocarbons are used.

2. The microencapsulation of concentrated hemoglobin solutions in phospholipid vesicles into so-called “artificial erythrocytes” is dealt with in Gaber et al., Encapsulation of Hemoglobin in Phospholipid Vesicles; Preparation and Properties of a Red Cell Surrogate in “The Red Cell Sixth Ann Arbor Conference”, G. J. Brewer (publisher), Alan R. Liss, Inc. New York, (1984), 179-190. This method, however, is still in the development stage of animal experiments at the present time. Moreover, the danger exists here of a lipoid overloading of the organism by the vesicle-forming lipids.

3. Preparation of suitable hemoglobin solutions. This method offers the best prospects of success.

The German Offenlegungsschrift 24 17 619 describes, by way of example, polymerized, combined hemoglobin as plasma protein substitute, dicarboxilidate-combined hemoglobin being prepared in the process.

The German Offenlegungsschrift 27 14 252 describes hemoglobin combined by pyridoxal phosphate.

The German Offenlegungsschrift 30 29 307 relates to a blood substitute, which is prepared by covalently bonding a polysaccharide, such as dextran, to cell-free hemoglobin.

The Belgian patent 838,933 describes the preparation of a water-soluble, combined, polymerized hemoglobin by reacting free hemoglobin with a polyfunctional combining agent and subsequently stopping the reaction with an inactivating agent. A polymeric hemoglobin with a molecular weight of 64,000 to 1,000,000 dalton is obtained.

U.S. Pat. No. 4,001,401 relates to a combined, polymerized hemoglobin as blood substitute and plasma expander with a molecular weight of 64,000 to 1,000,000 dalton, which is obtained by the combining agents glutaraldehyde, hexamethylene diisocyanate or butadiene diepoxide.

The European patent 0 201 618 relates to a method of preparing extremely high molecular weight, compact, soluble polymers, so-called hyperpolymers, of hemoglobin from highly concentrated solutions of monomeric hemoglobin.

In the German patent 37 14 351, this method is simplified in that erythrocytes can be used directly and the cross-linking agent no longer has to be added in a liquid phase.

For the preparation of suitable, modified hemoglobin solutions for routine clinical use, it is furthermore necessary to keep the viscosity of the solution as low as possible. The viscosity of the blood has a decisive influence on the so-called total peripheral resistance of the organism; the latter may not be excessively high, because this would not be tolerated by the circulatory system. In the case of polymer solutions, and this is true particularly also for hemoglobin polymers, such problems occur especially when a polymerization to chain molecules takes place. So that the viscosity of the solutions remains low, the polymer should be compact rather than an irrigated filamentary molecule, so that, while the viscosity of the plasma is as low as possible, the oxygen carrier can be applied in as high a concentration as possible. Einstein's viscosity law states that uniformly large spheres, independently of their radius, have a minimum viscosity.

Therefore, for reducing the viscosity, it would be extremely important to be able to prepare oxygen-carrying molecules of the utmost uniformity, as found, moreover, also in nature (earthworm). The molecular weight of the oxygen carrier could then be made very high, so that, on the other hand, the requirement of a negligible, colloidal, osmotic pressure could also be fulfilled.

In the event of a so-called hypocotic blood addition on the basis of hemoglobin solutions, it is moreover absolutely essential to remove particularly low molecular weight portions of the hyperpolymers.

The problem of combining hemoglobin into compact but soluble giant molecules can be regarded as having been solved with the European patent 0 201 618 and the German patent 37 14 351. However, the methods described there lead to a mixture of hyperpolymers with a broad distribution of molecular weights and a disproportionate increase in the viscosity as the concentration increases. Barnikol and Burkhard, Adv. Exp. Biol. Med., 248, (1989) 335-340.

### OBJECT OF THE INVENTION

An object of the present invention is a method to separate molecularly uniform hemoglobin hyperpolymers from a known hemoglobin hyperpolymer solution with hyperpolymers of different molecular weights.

### SUMMARY OF THE INVENTION

One way of achieving this object consists in subjecting a solution of hyperpolymeric hemoglobins of different molecular weights to an ultrafiltration process using a filter with a normal cut-off threshold of 10⁶ g/mol.

The methods named are known in the art as methods of separating such proteins, the size or weight of which is determined maximally by a quaternary structure, an the basis of their molecular weight. The upper molecular weight range is about 500,000 g/mol. The size of the hyperpolymeric hemoglobin exceeds that of the quartenary hemoglobin in up to (5-10)×100 times. This is also the upper limit of commercially obtainable marker proteins for determining molecular weights by comparison. It is well known that, in the case of particularly large proteins, the problem exists that these are frequently not separated as a whole. Instead, for previously largely unknown reasons, they disintegrate totally or partly so that, as a result, only subunits are obtained.

In contrast to the usually available proteins, the hemoglobin hyperpolymers, on which the invention is based, are giant molecules, which have only recently been synthesized for the first time and the size and weight of which, depending on the degree of polymerization, are a hundred to several hundred times those of the quaternary-structured hemoglobin molecule and which as stated above, have an upper molecular weight range of about 500,000 g/mol.

Surprisingly, it has now been found that fractions with a uniform molecular weight can also be separated from a mixture of such hyperpolymeric, giant, hemoglobin molecules by the known methods of ultrafiltration, fractional precipitation, chromatography and partially dissolution. According to preliminary and provisional analytical studies of the viscosity, light scattering and ultracentrifugation (Poetschke, Bamikol, Biol. Chem. Hoppe-Seyler, 373, (1992), 811; and Massenkeil, Kirste, Poetschke, Barnikol, Biol. Chem. Hopper-Seyler, 373, (1992), 798), these giant hemoglobin molecules are chain molecules. With respect to ultrafiltration, it would rather have been expected on the basis of general knowledge that passage of such giant molecules through the essentially circular pores of an ultrafilter would not lead to any molecular weight-specific separation if for no other reason than the chain character of the hemoglobin hyperpolymers. Such a point of view is also supported by the fact that it has not previously been possible to remove high molecular weight portions in the filtrate. Surprisingly, contrary to expectations, it was nevertheless found that, with the help of ultrafiltration, it is possible to remove the low molecular weight portions in the retentate from a crude polymer with a broad molecular weight range.

Since the preparation of hemoglobin hyperpolymers has succeeded only very recently, there is basically as yet little experience with respect to the physical and chemical properties of these giant molecules. Consequently, their behavior during the precipitation process also was not foreseeable and the discovered suitability of this method was a surprising result. With the help of a fractional precipitation, the high molecular weight portions, for example, can be removed from a hemoglobin hyperpolymer with a broad range of molecular weights.

In this field of chromatography also, there is as yet no experience in dealing with such giant molecules as the hemoglobin hyperpolymers. Because of the size and chain character, a successful molecular weight-specific separation was not to be expected with this method. Surprisingly, however, it was found that uniform, hyperpolymeric hemoglobins of different molecular weights can be obtained by means of chromatographic methods.

This actually known method of removing excess reactants and monomeric and oligomeric hemoglobin molecules by swiftly washing a freshly cross-linked and initially still insoluble hemoglobin hyperpolymer, can be combined with the remaining, aforementioned methods without any problems. Hemoglobin hyperpolymers, the components of which have molecular weight uniformity in the highest degree, can thus be obtained in an unexpectedly simple manner.

Pursuant to an advantageous embodiment of the inventive method, different filter types are used for the ultrafiltration process or processes and/or multiple filtration takes place and/or the pH is changed and/or ultrafiltration is carried out at different concentrations and/or the composition of the solvent is varied and/or the temperature is varied and/or the transmembrane pressure and/or the tangential (over)flow is varied.

Advantageously, for the fractional precipitation processes, different agents can be used and/or the precipitation can take place at different temperatures and/or the pH can be varied and/or different solvents can be used for the polymeric hemoglobins that are to be precipitated and/or the reaction times can be varied and/or different, saturated precipitating agents can he used.

In a preferred embodiment, different chromatographic methods, particularly ion exchange and gel filtration chromatography are used in combination or successively for the chromatographic process or processes, and/or the pH is changed and/or the composition of the solvent is varied and/or the chromatographic conditions, particularly the flow and the load, are varied and/or different chromatographic materials are used.

For the partial dissolving process or processes, provisions are preferably made so that the dissolving time is varied and/or at least one washing is carried out and/or the dissolving polymers are treated with stabilizing agents, which prevent degradation of the polymers especially due to oxidation, and/or the dissolving conditions, particularly the temperature and/or the amount of solvent, are changed and/or the composition of the solvent is varied.

By varying the different parameters, it is possible to match each of said processes to the nature (e.g., different cross linking agents and/or different hemoglobins, such as bovine, porcine or human hemoglobin) of the particular hemoglobin hyperpolymer mixture, which is present and has to be separated, and to achieve an advantageously high separation effect for hemoglobins with one molecular weight within very narrow limits. Even if the different stabilities of the various hyperpolymeric hemoglobins is taken into consideration, a combination of the different separation processes with one another enables hemoglobin hyperpolymers to be prepared, the molecular weights of which are uniform in the highest degree.

The invention is described in greater detail in the following by means of some examples, it is understood that these examples are provided by way of illustration and not by way of limitation.

### EXAMPLE 1 (ULTRAFILTRATION)

Human hemoglobin was cross linked with the help of glutardialdehyde to hyperpolymers in accordance with a known protocol (Poetzschke, Barnikol, Biomater. Art. Cells and Immob. Biotechn., 20, (1992), 287-291). An approximately 20% solution of the hyperpolymers was then subjected to an ultrafiltration in the BIKU electrolyte solution (in mmole/L: NaCl 125; KCl 4.5; NaHC03 20; 0.2 g/L of NaN3). A filter with a normal separating limit of 106 g/mol was used. The filtration process was carried out with a liquid volume, approximately twenty times that of the original solution.

The hyperpolymeric hemoglobins can be analyzed with respect to their polymolarity with Sephacryl S-400 HR (Deutsche Pharmacia, Freiburg, Germany). The boundary molecular weights of the unfiltered product were 65,000 (bottom) and 15×106 g/mol (top); after the ultrafiltration, those of the retentate were 500,000 and 15×106 respectively. With the help of ultrafiltration, it has thus been possible to remove the low molecular weight portions from the crude polymer, the components of which covered a very broad range of molecular weights, and thus to lower the colloidal osmotic pressure of the polymer decisively.

### EXAMPLE 2 (FRACTIONAL PRECIPITATION)

Bovine hemoglobin was polymerized with the bifunctional cross-linking agent, 2,5-diisothiocyanate benzenesulfonate (DIBS) by known methods (W. K. R. Barnikol, Adv. Exp. Biol. Med., 1993, in print) to a hyperpolymer with a very broad molecular weight distribution.

For the precipitation, a 4-molar (saturated) solution of ammonium sulfate, the pH of which was adjusted with a 25% ammonium hydroxide solution to a pH of 7.3. A 3.5% solution (0.8 mL) of the above-named hemoglobin hyperpolymer and 0.5 mL of the 4-molar ammonium sulfate solution were mixed and allowed to stand for 4.5 hours at room temperature. This was followed by sharp centrifuging and pipetting off the supernatant.

A comparison by gel chromatography on Sephacryl S-400 HR (Deutsche Pharmacia, Freiburg, Germany) revealed a lower molecular weight of 65,000 and an upper molecular weight of 15×106 g/mol for the unfractionated sample. On the other hand, values of 65,000 and 940,000 g/mol respectively were found in a sample of the supernatant. The high molecular weight portions have thus been removed successfully from a hemoglobin hyperpolymer with a broad molecular weight range.

### EXAMPLE 3 (CHROMATOGRAPHY)

The starting material is a DIBS polymer prepared in Example 2. The preparative fractionation was carried out on Sephacryl S-400 HR with HeNa as solvent (144 mmoles/L of NaCl; 10 mmoles/L of HEPES buffer, 0.2 g/L of NaNj), column: 2.6 cm diameter, capacity 510 mL, flow rate 27 mL/hour. A 2.5% hyperpolymeric hemoglobin solution (10 mL) was applied and the fractions were analyzed by gel chromatography as described in Example 2.

The molecular weight of the starting material ranged from 65,000 to 15×106 g/mol and that of the fractions obtained ranged from 215,000 to 1.05×106 g/mol and from 65,000 to 0.32×106 g/mol. This example shows that chromatographically uniform hyperpolymeric hemoglobins of different molecular weight can be obtained.

### EXAMPLE 4 (PARTIALLY DISSOLVING)

Human hemoglobin was cross linked with glutardialdehyde, as described in Example 1. After the sample was divided, (a) the precipitate was dissolved for 24 hours and (b) for only 30 minutes. In both cases, the supernatant was obtained and analyzed by gel chromatography, as described in Example 1. In case (a), the molecular weights extended from 65,000 to about 15×106 g/mol and in case (b) on the other hand from 65,000 to 3.6×106 g/mol.

This example shows the possibility of obtaining hemoglobin hyperpolymers of low molecular weight by a dissolving process of appropriate duration.

1. A method of preparation of molecularly uniform Hyperpolymeric gemoglobins, comprising the steps of providing a filter with a normal cut-off threshold of 10⁶ g/mol; and subjecting a solution of hemoglobin hyperpolymers of a different molecular weight, to an ultrafiltration process using the filter.

2. A method according to claim 1, wherein the subjecting step includes a solution of hemoglobin polymers to infiltration process to obtain hemoglobin polymers with a final molecular weight in a range from about 500,000 to 15×10⁶ g/mol.
